(19) United States
(12) Patent Application Publication
(10) Pub. No.: US 2004/0044307 A1
(43) Pub. Date: Mar. 4, 2004

(54) Urinary catheters

(76) Inventors: Margaret P. Richardson, Saundersfoot (GB); Philip Richardson, Saundersfoot (GB)

Correspondence Address: NIXON & VANDERHYE, PC, 1100 N GLEBE ROAD, 8TH FLOOR, ARLINGTON, VA, 22201-4714, US

(21) Appl. No.: 10/398,461

(22) PCT Filed: Oct. 4, 2001
(86) PCT No.: PCT/GB01/04465

(30) Foreign Application Priority Data
Oct. 5, 2000 (GB) 0024355.0

(51) Int. Cl. A61M 29/00

(52) U.S. Cl. 604/102.01; 604/544

(57) ABSTRACT

A urinary catheter of the type which is retained in the urethra by means of a locating balloon also includes an inflatable valve by means of which the lumen (3) of the catheter may be opened or closed by the user. The catheter is manufactured as a two or three layered tube: the outer layer (1) includes an inflatable portion (5) for forming a locating balloon, whilst the inner layer (2) includes a further inflatable portion (6) providing the internal valve. A supply of fluid, e.g. saline, is fed to the inflatable portions, via relatively small diameter tubing encased in the laminar catheter structure. The pressure of the fluid is controlled by a manual pressure control device (19, 29).

This invention relates to urinary catheters, and in particular urinary catheters which are retained in the body for a period of time.

Urinary catheters are often inserted as a result of injury or medical condition, or because, as the body ages, the action of the bladder deteriorates. Urinary catheters are also used before or after surgery on the bladder, prostate or any other part of the urinary tract.

Urinary catheters are available in a variety of sizes and lengths to suit various applications such as a for a male adult, female adult or child.

Until the 1930's, all catheters were of the intermittent type and could not be left in the bladder permanently. However, the development of a urinary catheter by Dr. Frederick Foley which had a locating balloon enabled the catheter to be retained in the bladder and to drain continuously into a bag carried either by the patient attached to the leg, or attached to the patient's bed.

The most commonly used catheters are currently the Foley catheters, which comprise two concentric tubes, the inner tube wall and the outer tube wall being substantially joined together with a limited length of the outer tube wall of the catheter having a relatively thinner wall section not joined to the inner tube wall and capable of being inflated to form a locating balloon. The inner tube wall of the conventional Foley catheter is so constructed as to retain its substantially circular cross-section and thus remain open when subject to continuous passage of urine from the bladder.

When a Foley catheter is inserted into the bladder via the urethra, the section of the outer tube wall not joined to the inner tube wall is inflated with saline solution to form the locating balloon arrangement within the bladder whilst the inner tube allows the continuous passage of urine from the bladder to an external collection bag. The balloon is inflated with saline solution by means of a small diameter connector tube moulded within the catheter and terminating in a mechanical filling and emptying valve outside the body. A syringe is used to inflate the balloon with saline solution after the catheter is installed and deflate the balloon when removal of the catheter is required.

Patients who have Foley Catheters installed, particularly on a long term basis, are at risk of complications, such as urinary tract infections resulting from bacteria gaining entry to the bladder via the catheter lumen, as well as the risk of tissue damage and encrustation of the catheter (which may therefore cause blockage). The need to continuously connect a Foley catheter to a receiving bag in order to permit collection of urine can cause considerable inconvenience to the patient, as well as the risk of infection.

We have now devised an arrangement which overcomes some of the problems outlined above.

WO99/51293 describes a urinary catheter having a magnetic valve which is electrically connected to a control unit which controls the magnetic valve which is arranged in a channel for carrying away urine. However, the described arrangement is relatively complicated as well as requiring continuous connection to a power supply.

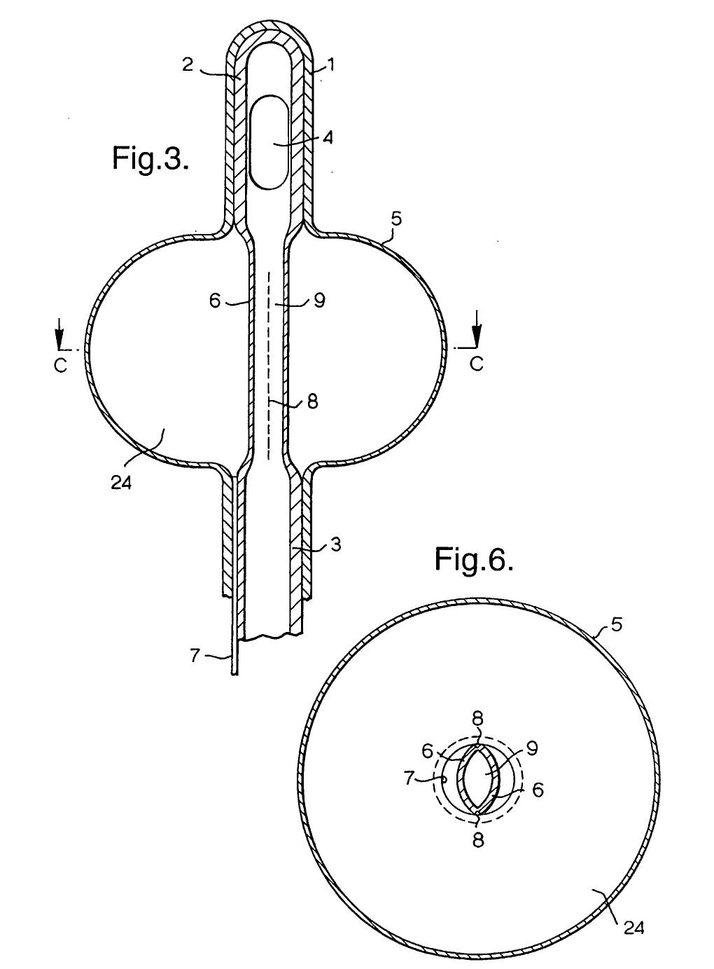

WO00/00247 describes a urinary catheter with an enlarged head in which is located an electromagnetic valve. It is not clear how this catheter would be installed in the user since the enlarged head portion does not appear to be deflatable. WO00/00247 also briefly mentions the use of a hydraulic or pneumatic valve for closing the lumen of the catheter; this arrangement is shown in FIGS. 3 and 4. The valve balloon is located proximally of the retaining balloon (ie. further away from the bladder). There is no disclosure as to how this arrangement is constructed.

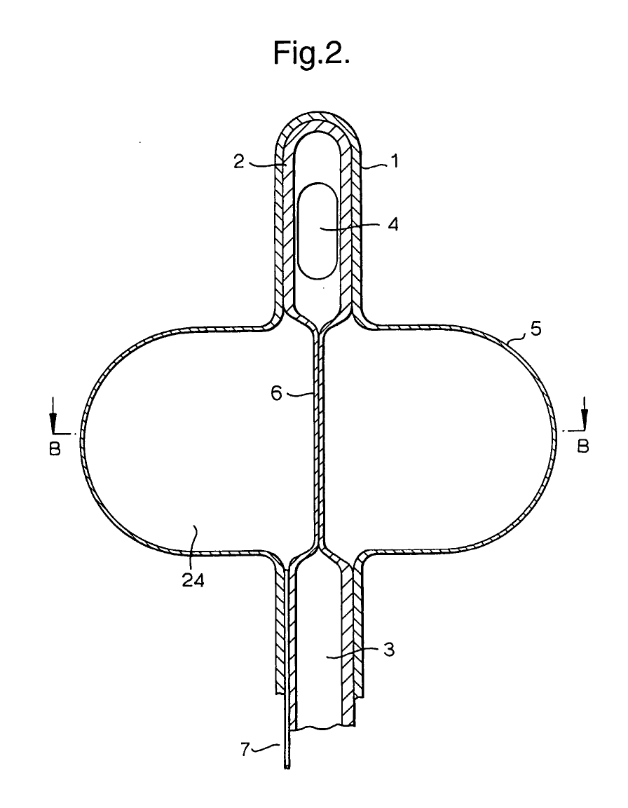

U.S. Pat. No. 6,102,848 describes a urethral valve system which, in one embodiment (FIGS. 2a and 2b) employs a retaining balloon 22 through which is a channel to allow urine to pass through it. Within the channel is a valve balloon 12, which is structurally separate from the retaining balloon and which is by some means activated by a control line 14 extending down the urethra to a point external to the body. This device is a urethral valve system as opposed to a urinary catheter. Its construction and operation are a little unclear, but it can be envisaged that manufacture of a double balloon arrangement of this sort would present problems.

In accordance with a first aspect of the present invention a urinary catheter comprises:

a) a first tube or part-tube;

b) a second tube exterior of and concentric with the first tube or part-tube and joined to it for part of the area of the first tube or part-tube;

c) the first tube or part-tube having a first inflatable portion to which the second tube is not joined;

d) the first inflatable portion being inflatable such as to occlude the lumen of the catheter.

Preferable features of the first aspect are set out in the accompanying claims.

In accordance with a second aspect of the present invention, there is provided a urinary catheter comprising at least two concentric tubes, an inner tube wall and an outer tube wall being partially joined together with a length of the outer tube wall of the catheter having at least a section not joined to the inner tube wall and capable of being inflated to form a locating balloon, the urinary catheter including control means for selectively controlling the flow of urine from the urinary bladder, when in use.

In other words, the second aspect of the present invention provides a modified Foley catheter including control means for selectively controlling the flow of urine from the urinary bladder, when in use.

The control means preferably comprises hydraulic or pneumatic control means. In accordance with a third aspect of the present invention there is provided a urinary catheter including pneumatic or hydraulic control means for selectively controlling the flow of urine from the urinary bladder, when in use.

The catheter is preferably a modified Foley catheter i.e. of the type having two concentric tubes the inner tube wall and the outer tube wall being partially joined together with a length of the outer tube wall having at least a section not joined to the inner tube wall and being capable of being inflated to form a locating balloon.

By providing means for controlling the flow of urine from the bladder, as opposed to allowing a continuous passage of urine from the bladder, as in the conventional Foley catheter, the patient can be given full control over the emptying of his/her bladder, thereby eliminating the need to carry a urine collection bag. Not only is this more convenient and comfortable for the patient, but it also reduces the risk of infection caused by urine retention in the catheter tube. Furthermore, no power supply is required to control of flow of urine in the modified Foley catheter and the resulting arrangement can be relatively simple and cost effective.

Thus, the catheter of the invention preferably comprises a tube structure for insertion in a urinary bladder, the tube structure comprising an inlet for receiving urine, an outlet for expelling urine and means for retaining the catheter in the bladder, in use. The tube structure beneficially comprises an inner tube and an outer tube, both of substantially flexible material, the inner and outer tubes being bonded together at the distal end (to be inserted into the bladder, in use), with the inlet comprising an aperture.

Corresponding portions of the walls of the inner and outer tubes typically have reduced thickness, at which areas the tubes of the modified Foley catheter of the invention typically not bonded together, substantially the remaining portion of the inner and outer tubes typically being bonded together, so that the non bonded areas form an annular space. There may be beneficially provided a relatively small opening, which preferably cooperates with a tube or the like, for introducing fluid, preferably a liquid such as saline solution, into the annular space, so as to cause the outer tube portion to expand into a balloon-like structure and thereby retain the catheter in the bladder. Unlike the conventional Foley catheter, where the inner tube is constructed to remain open to allow continuous flow of urine when the balloon-like structure is inflated, the inner tube of the modified Foley catheter of the invention is constructed to collapse and prevent the flow of urine.

In use, fluid pressure within the annular space causes the inner tube portions not bonded to the outer tube . portions to collapse structurally so that under a predetermined level of pressure, the inner tube entirely collapses structurally and blocks the tube, thereby preventing urine from exiting the bladder.

It is preferred that the inner tube wall in the corresponding portions of the inner and outer tubes not bonded together be of reduced thickness of section. Thus, it is preferred that the reduced thickness of section of the inner tube wall is such that it forms a valve, preferably a hydraulic valve, which when subject to increasing pressure on its surface will at a relatively precise pressure cause the inner tube to collapse on itself. The collapsed inner tube closes the inner tube and prevents the flow of urine from the bladder i.e. the valve is closed.

It is preferred that the reduced thickness of section of the inner tube wall is such that decreasing pressure will at a relatively precise pressure, cause the collapsed inner tube to at least partially re-form its un-collapsed shape. When the inner tube re-forms, the inner tube is opened and allows the flow of urine from the bladder i.e. the valve is open.

The control means beneficially includes means for reducing the fluid pressure in the annular space, so as to at least partially reverse the structural collapse of the inner tube and provide a passage for the flow of urine from the bladder. The control means preferably also includes means for re-introducing fluid into the annular space to increase the pressure and re-collapse the inner tube to once again prevent the flow of urine from the bladder.

It is preferred that the valve and the locating balloon may be inflated and partially deflated simultaneously during emptying of the bladder and opening and closing of the valve.

It is preferred that the valve and locating balloon be located at or near the distal end, i.e. the bladder end, of the catheter.

It is preferred that the region of inner tube of reduced thickness wall be scored i.e. partially cut through the wall thickness with one or more scores (preferably two such scores).

The scores or each score located on the inner tube wall provide hinge points so that when subjected to increased hydraulic pressure the two or more segments forming arched sections of the inner tube will collapse fully to completely seal the inner tube.

It is further preferred that two partial cuts or scores be provided in the external surface of the inner wall, beneficially in the longitudinal direction of the catheter.

It is preferred that two partial cuts be provided in the surface of the inner tube wall, spaced 180 degrees apart across the tube section.

It is preferred that the valve is subjected to varying pressure by the provision of a relatively small diameter connecting tube, preferably connected to an external pressure adjusting control device.

In one embodiment, the external pressure adjusting control device for the hydraulic valve may be located at the proximal end of the urinary catheter external to the body of the patient.

The external means for temporarily reducing the hydraulic pressure to operate the hydraulic valve may be integrated with a mechanical balloon inflation valve.

Thus, in a first embodiment of the invention, the non-bonded portion of the inner tube wall which may be of reduced thickness of section (the preferably hydraulic valve) is preferably adjacent to the non-bonded portion of the outer tube wall, which may be of reduced thickness of section such that when the annular space between the two tube walls is filled with, for example, saline solution under pressure, the outer tube will distend to form a locating balloon whilst the inner tube will structurally collapse to seal the inner tube or lumen and prevent the flow of urine from the bladder.

A small diameter connecting tube which is connectable to an external pressure adjusting control device may be provided to supply saline solution or the like to the annular space between the tube walls in order to fill it with saline solution or the like under pressure, in order to substantially simultaneously inflate the locating balloon and close the hydraulic valve.

It is further preferred that the small diameter connecting tube which is connected to an external pressure adjusting control device may also remove the saline solution (or the like) from the annular space between the two tube walls in order to open the valve.

If the pressure in the system is lowered, the balloon may reduce in diameter. The resultant reduced pressure allows the inner tube to partially reform its original section. Thus the valve and lumen are open and urine may flow from the bladder. This arrangement constitutes an inner tube wall hydraulically operated closure or valve which can be operated by varying the hydraulic pressure applied to the outer tube wall balloon.

The external means for temporarily reducing the pressure within the hydraulic system controlling the valve may be under direct patient control. Thus when emptying of the bladder is required, the patient can reduce the hydraulic pressure in the system manually. This allows the collapsed section of inner tube forming the hydraulically operated valve to reform into an open tube, thus permitting the flow of urine from the bladder out through the lumen of the catheter in the normal way. Urination will then be under the direct control of the patient via the hydraulically operated artificial valve and the need for a collection bag is obviated as normal function of the bladder is effectively restored.

The preferably hydraulically operated valve may be re-closed by the patient restoring the original hydraulic pressure to the system thus re-collapsing and sealing the region of inner tube of reduced wall thickness.

In the first embodiment of the invention, the need for a collection bag. is alleviated, but it is preferred that a spigot or the like be provided to seal the external end of the catheter when the bladder is not being drained. This will prevent contamination through the catheter lumen and act as a ‘plug’ or secondary valve in case of failure of the primary hydraulic catheter valve.

Optionally, the modified Foley catheter of the present invention may be temporarily connected to a conventional drainage bag if required, for example, overnight. In use, the spigot would be removed, the drainage bag connected in a conventional manner and the valve opened as described above to enable a continuous flow of urine into the bag.

In a second embodiment of the invention, the preferably hydraulic valve is arranged to be actuated independent of the locating balloon via its own small diameter connecting tube to an external pressure adjusting device. In this embodiment, there are two, preferably hydraulic, tubes, one for inflating the locating balloon and an independent hydraulic tube for operation of the hydraulic valve.

In this embodiment, the catheter preferably comprises three or more concentric tubes, the outer two tubes preferably being arranged to form the locating balloon, in use, in the conventional manner as described with reference to the conventional Foley catheter. The third internal tube is preferably substantially bonded to the second tube, with an area of the third internal tube not bonded to the second tube preferably forming the control valve which, when inflated, is capable of closing the lumen and thus inhibiting the flow of urine from the bladder. This control valve is preferably supplied via its own small diameter connecting tube from an external pressure adjusting device. In this embodiment of the invention, the valve controlling urine flow may be operated independently of the locating balloon.

In this embodiment, it is preferred that the area of the third internal tube not bonded to the second tube extends over a substantial length of the catheter, the non-bonded area preferably being located adjacent to the distal end of the catheter. It is further preferred that the third internal tube has a thinner section than the second tube, and the second tube is preferably of sufficient thickness in section to remain structurally stable when the control valve is inflated and when the locating balloon is inflated.

Thus in this embodiment of the invention, the hydraulic valve is arranged to be actuated independently of the locating balloon, preferably via its own small diameter connecting tube to an external pressure adjusting device. In fact, in this embodiment of the invention, two hydraulic tubes are preferably provided, one for inflating the locating balloon and one for independent generation of the hydraulic valve.

It is preferred that the external means for temporarily reducing hydraulic pressure to operate the hydraulic valve or the locating balloon be integrated with a mechanical balloon inflation valve similar to that usually installed on a conventional Foley catheter.

In either embodiment, the catheter is provided with a hydraulically operated valve and means for external control, as described above, and may be further modified by shortening the overall length of the catheter (relative to the length of a conventional Foley catheter) so that it terminates within the urethra.

In this modification, an hydraulic tube or tubes of diameter substantially less than the urethra extend from the hydraulic valve and balloon arrangement down the urethra to the exterior of the body to a pressure controlling device which operates the hydraulic valve. In this modification urine passes through the shortened catheter and down the urethra. The patient has full control over bladder function as well as a regular flow of urine flushing through the urethral tract.

It is preferred that the external means for temporarily reducing the hydraulic pressure to operate the hydraulic valve or the locating balloon be integrated with a mechanical balloon inflation valve.

It is also preferred that the pressure controlling device for the catheter described in any one of the embodiments above is located at the distal end of the tube controlling the valve and/or inflation of the locating balloon.

It is further preferred that the pressure controlling device encompasses means for initially inflating the locating balloon with saline solution. This can be provided, for example, by a one way mechanical valve connector device of the type commonly used with conventional Foley catheters.

It is further preferred that the pressure controlling device is in the form of a clam shell-like case squeezing a region of the tube preferably having locally increased diameter. Thus, when the clam shell-like case is closed onto the increased diameter tube, the volume of the tube is reduced and consequently the pressure in the closed system is raised. With the clam shell-like case squeezing the tube the volume of the locating balloon is maximised and the increased hydraulic pressure within the system causes the hydraulic valve to close. That is, it causes the inner tube in the region of the balloon to collapse, thus sealing the tube.

The clam shell-like case is preferably restrained in the closed position, that is, squeezing the hydraulic tube, by the provision of a retaining clip or similar device.

It is further preferred that the region of tube squeezed by the clam shell-like case is dimensioned and of such flexible material that removal of the squeezing effect of the clam shell-like case, that is when the clam shell-like case is opened, has the effect of restoring the original relaxed (non-squeezed) shape of the tube thus increasing its internal volume relative to the squeezed state.

The increased volume of the relaxed tube has the effect of reducing pressure in the hydraulic system allowing the inner tube in the region of the balloon to reform its shape, thus opening the lumen of the tube and allowing urine to flow, ie. the hydraulic valve is opened.

It is preferred that the clam shell-like case be capable of being opened and closed by the user, thus effecting external control of the hydraulic valve.

It is further preferred that the clam shell-like case and region of hydraulic tube of increased diameter should be attached together by adhesive, mechanical means or similar.

A fourth aspect of the invention is considered to reside in a pressure control device suitable for controlling hydraulic or pneumatic pressure in a urinary catheter, e.g. of Foley type, having a hydraulic or pneumatic valve, the said pressure control device having any combination of the above described features.

A fifth aspect of the invention resides in an alternative pressure control device, comprising a syringe device for inflating and deflating the hydraulic or pneumatic valve of a valved urinary catheter. Preferably the syringe device is biased into a closed position with the plunger depressed and is movable, and preferably lockable, against the bias force into an open position with the plunger partially withdrawn. Preferably, a line to a urinary catheter locating balloon is connected to the syringe device and one or more inlet ports provided in the syringe device via which the locating balloon and/or the syringe chamber may be filled. Preferably, a third position of the plunger is possible in which the said line is open to the syringe chamber whereby filling of the locating balloon and syringe chamber may be effected via a single inlet port.

It will be appreciated that the various preferred or optional features of the second and third aspects are equally applicable to the first aspect of the invention.

The present invention offers the following main benefits to the patient over existing urinary catheters:

(1) the catheter can effectively restore normal bladder function by returning the control of such function to the user, in an easy to operate manner;

(2) the catheter can reproduce the action of the human bladder valve;

(3) the bladder valve can be self cleansing and under manual control;

(4) the catheter can alleviate the risks associated with reflux of urine into the bladder;

(5) the catheter can reduce the risk of infection which otherwise occurs between the catheter wall and the urethra;

(6) the catheter can remove the necessity for the user to carry a urine collection bag;

(7) the catheter can permit restoration of dignity to the patient.

In addition, the catheter of the present invention and its valve arrangement can be fail-safe and unlikely to cause damage to the patient through failure. It can be simple and inexpensive to manufacture and can use existing materials and technology.

Embodiments of the present invention will now be described by way of example only, and with reference to the accompanying drawings, in which:

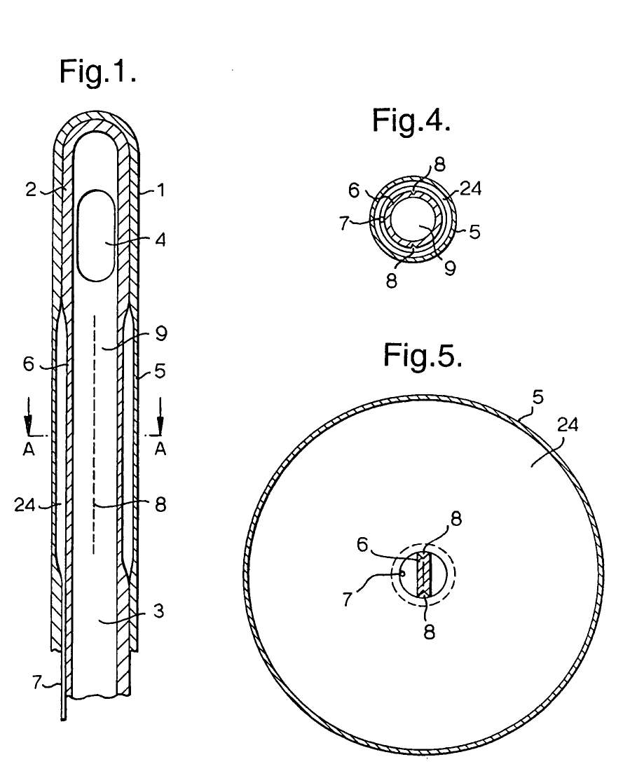

FIG. 1 is a longitudinal section through a urinary catheter in accordance with a first exemplary embodiment of the present invention;

FIG. 2 is a longitudinal section through the urinary catheter of FIG. 1 showing the locating balloon inflated and the hydraulic valve closed;

FIG. 3 is a longitudinal section through the urinary catheter of FIG. 1 showing the locating balloon partially inflated under reduced pressure enabling the hydraulic valve to open allowing urine to pass down the lumen;

FIG. 4 is a cross section of the urinary catheter at A-A in FIG. 1;

FIG. 5 is a cross section of the urinary catheter at B-B in FIG. 2;

FIG. 6 is a cross section of the urinary catheter at C-C in FIG. 3

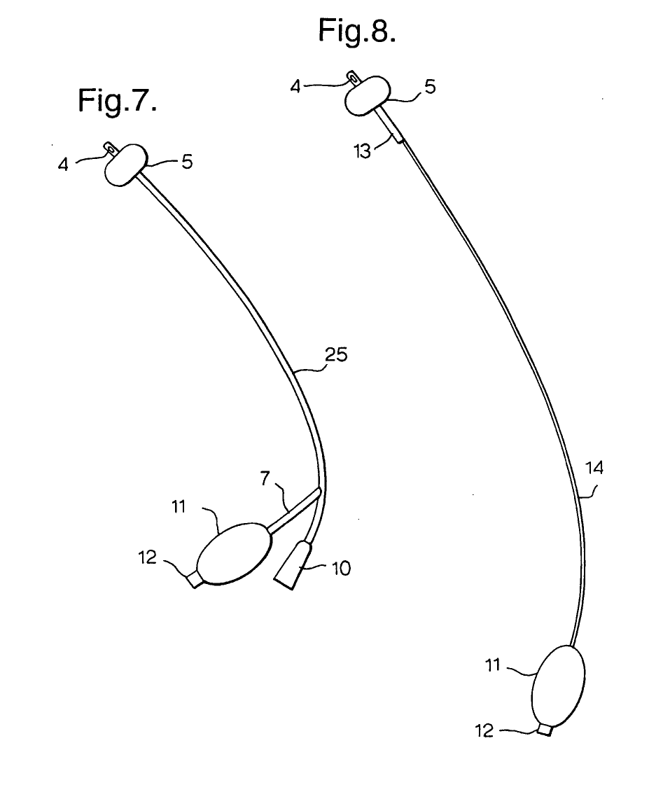

FIG. 7 is a general arrangement diagram of a urinary catheter according to a first exemplary embodiment of the invention, showing the inflated locating balloon and external control device;

FIG. 8 is a general arrangement diagram of a urinary catheter according to another exemplary embodiment of the invention, showing the inflated locating balloon and external control device;

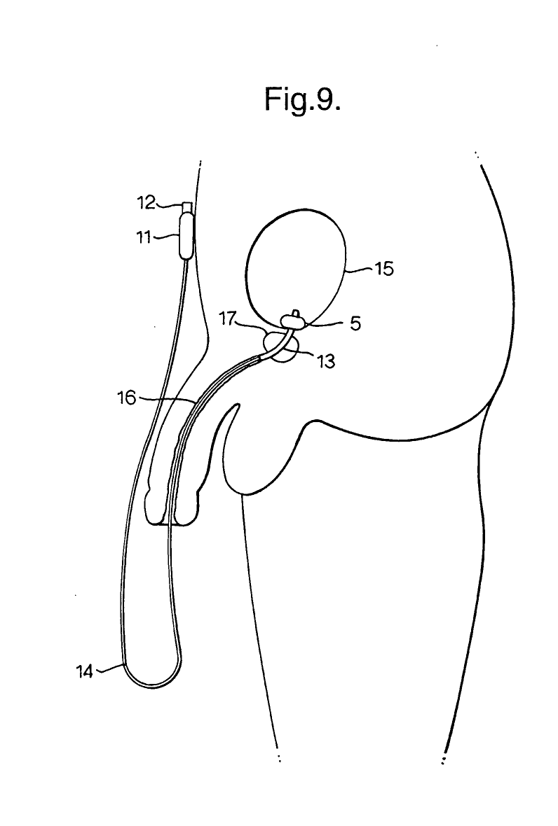

FIG. 9 is a general arrangement diagram of the urinary catheter of FIG. 8, when installed in a male patient;

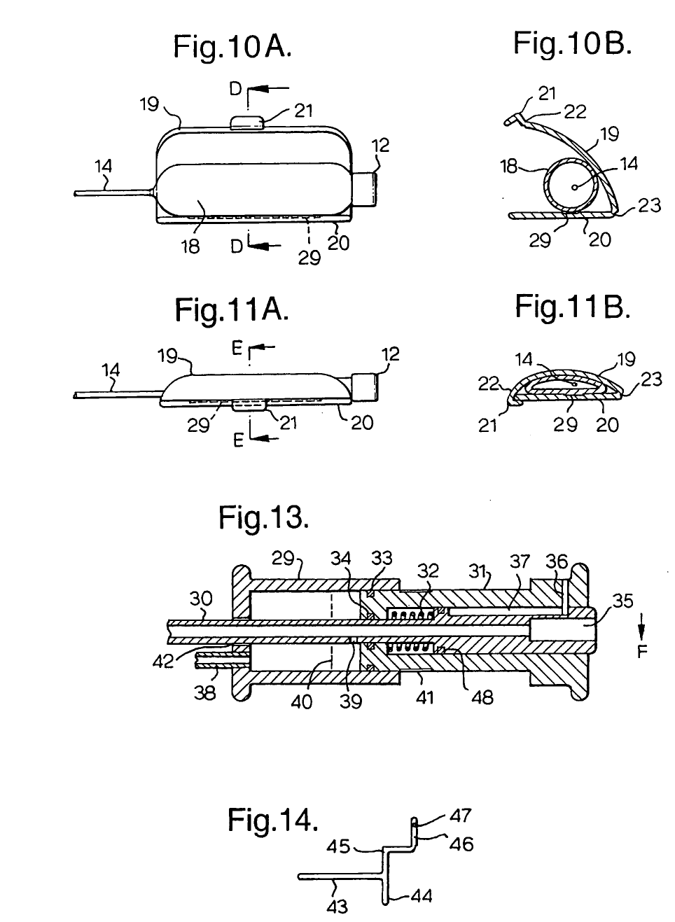

FIGS. 10a and b are an elevation and cross section respectively, of an external control device for use in a catheter according to an embodiment of the invention, the control device being shown in the open position allowing the hydraulic valve to open and urine to flow;

FIGS. 11a and b are an elevation and cross section respectively, of the external control device of FIG. 10, shown in the closed position keeping the hydraulic valve closed and preventing the flow of urine;

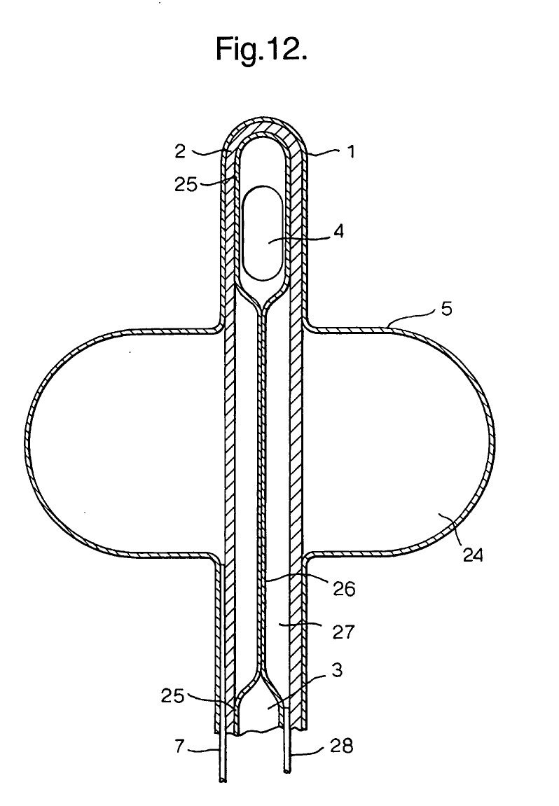

FIG. 12 a longitudinal section through a urinary catheter according to a second exemplary embodiment of the invention;

FIG. 13 is longitudinal section through an alternative control device for use in a catheter according to the invention; and

FIG. 14 is a view in the direction F of the slot 37 of FIG. 13.

FIG. 1 shows a detail of the longitudinal section through the urinary catheter at the distal end which is inserted into the bladder. In use, the urinary catheter comprises an inner tube 2 and an outer tube 1, both of substantially flexible material, forming a closed end. Urine can enter the inner tube or lumen 3 through an aperture 4 adjacent to the distal end. Over a limited length of the catheter, the outer tube wall thickness is reduced 5. Over the same length of catheter the inner tube wall thickness is also reduced 6. The inner and outer tubes are not joined together over the lengths of reduced wall thickness, but the rest of the inner and outer tubes are substantially bonded together to form a seal, such that the reduced thickness areas form an annular space 24. A connecting tube 7 of relatively small diameter cooperates with the annular space 24 between the inner and outer tubes to allow fluid, such as saline solution, to be pumped into it. Also shown is the location of two scores 8 on the outer surface of the inner tube 6 on a line parallel with the longitudinal axis.

It is possible to envisage a number of modifications to this design which would achieve essentially the same effect. For example, the outer tube 1 need not have a portion 5 of reduced thickness, even though this is the more desirable construction. What is required is that the portion 5 be expandable at a given pressure in the annular space 24, and that this pressure 24 is not sufficient to cause occlusion of the lumen 3 by distortion of the inner tube 2. It could be envisaged that the outer tube could have a region corresponding to the region 5 in FIG. 1 which, instead of or in addition to having reduced thickness, has a reduced modulus of elasticity or has lines of weakness or areas of weakness formed in it. In this embodiment, where the inner and outer tubes form the boundaries of a single annular space 24 into which pressurised fluid is injected, it is necessary that the inner tube does not deform substantially until the outer tube is fully deformed or inflated. It will be appreciated that the same possibilities as those discussed above therefore also apply to the design of the inner tube, that is to say using a material of different elastic modulus, either for all of the first tube or just the inflatable portion 6, or alternatively forming areas or points or lines of weakness in the inner tube. It is also possible that the inner tube may be designed to inflate, deform or collapse over only part of its circumferential extent; for example the tube could be made thinner only on one side so that, at an appropriate pressure, one side of the tube bulges inwardly and seals against the opposite side of the lumen.

It will be appreciated that it is not necessary for the deformable or inflatable portions 5, 6 of the outer and inner tubes to be completely coincident as illustrated in FIG. 1. These regions may only partially overlap in the longitudinal direction and, indeed, may only partially overlap around the circumference (for example as would be the case if only one side of the inner tube were inflatable). It would also be possible for the inflatable portions 5, 6 not to overlap at all, in which case they may still be inflatable from a common source of pressure if the connecting tube 7 divides at some point in order to separately supply the two inflatable portions.

Referring now to FIG. 2, fluid such as saline solution is pumped through the connector tube 7 at increasing pressure and accumulates in the annular space 24, thereby causing the relatively thin outer tube 5 to expand like a balloon. The increase in diameter in this part of the catheter holds it in position within the bladder of the user. Under the increased hydraulic pressure within the annular space 24 the length of relatively thin inner tube 6 structurally collapses thus closing the lumen 3 and preventing urine from leaving the bladder of the user.

Referring to FIG. 3, when the user requires to urinate, a lowering of the hydraulic pressure of the saline solution within the annular space 24 allows the length of relatively thin inner tube 6 to partially reform its original shape, thus opening the lumen 3 and allowing urine to flow from the bladder of the user down the lumen 3. The scores 8 in the outer surface of the inner tube allow the inner tube in this section to collapse or re-form at relatively precise hydraulic pressure to seal the lumen 3 and thus prevent urine flow or to open the lumen 3 and allow urine flow. Thus the combination of thinned wall section and scoring of the inner tube allows this length of inner tube to act as a valve controlled by varying the pressure within the annular space 24 of the locating balloon.

FIG. 4 shows a cross section A-A through the annular space 24 between the outer tube at 5 and inner tube at 6, together with the hydraulic connector tube 7 through which fluid such as saline solution may be pumped into the annular space 24 to inflate the locating balloon. Also shown are the two scores 8 placed diametrically.opposite each other on the inner tube at 6 which form elastic hinge points enabling the collapse of the inner tube and thus closure of the lumen 3 to occur at a relatively precise hydraulic pressure in the annular space 24.

Referring to FIG. 5, there is shown a cross section B-B when the annular space 24 is filled with fluid such as saline solution under pressure supplied from connecting tube 7. The relatively thin outer tube wall 5 is distended forming the locating balloon for the catheter. The relatively high hydraulic pressure causes the relatively thin walled inner tube at 6 to collapse with the scores 8 in the outer surface of the inner tube 6 acting as hinges to enable the two halves of the inner tube at 6 to collapse to a flat section so as to substantially totally seal the lumen at 9.

Referring to FIG. 6 of the drawings, there is shown a cross section at C-C. The hydraulic pressure within the annular space 24 has been reduced by drawing the fluid such as saline solution from the annular space 24 via the connector tube 7 using an external device (not shown) which is under the control of the user. The degree of distension of the balloon is reduced as the outer tube 5 reduces in diameter. With the reduced hydraulic pressure the inner tube 6 is allowed to partially re-form its un-collapsed shape. The lumen 9 is partially opened enabling normal urination to take place under the direct control of the user. After urinating the user using the external control device pumps the saline solution back into the annular space 24 via the connecting tube 7 which increases the hydraulic pressure within the annular space 24 re-inflating the balloon fully and re-collapsing the inner tube at 6 to reseal the lumen at 9, thereby preventing the flow of urine from the bladder.

Referring to FIG. 7, the general arrangement of the assembled urinary catheter is shown. At the distal end of the catheter comprising the aperture and inflated locating balloon 5 is the part of the catheter normally located in the neck of the bladder of the user. The tube of the catheter 25 comprises the inner tube for carrying urine to the discharge point 10 and a connection tube 7 which branches from the main catheter tube 25 at the proximal end of the catheter and terminates in the control device 11 outside the body of the user. The initial inflation of the locating balloon 5 and its final deflation to enable removal of the catheter is performed with a syringe (not shown) connected to the port 12 in the way that conventional Foley catheter balloons are usually inflated and deflated.

It will be noted that the part of the catheter which carries urine terminates well outside the body. The discharge point may be provided with a stopper, spigot, cap or similar (not shown), to form a secondary seal to further reduce the possibility of bacteria travelling through the lumen of the catheter from the outside of the body and also to act as a secondary valve to prevent inadvertent urine discharge in the event of failure of the hydraulic valve at the distal end of the catheter.

FIG. 8 of the drawings shows an alternative arrangement according to another exemplary embodiment of the invention, where the section of catheter carrying urine 13 is truncated so that when installed in the user, the catheter discharges urine within the urethra. Normally the truncation point of the catheter 13 would be just after the prostate gland in males. The small diameter connecting tube 14 passes down the urethra to the outside of the patient's body terminating in the control device 11. The benefit of this arrangement is that normal urethral function, and in particular the beneficial flushing action during urination for the control of bacterial infection is restored.

Referring to FIG. 9, the shortened version of the urinary catheter (as shown in FIG. 8) is shown installed in a male user. It can be seen that the distal tip of the catheter with its locating balloon 5 is installed in the neck of the bladder 15. The drawing also shows the short urine carrying tube 13 terminating just after the prostate gland 17 enabling the urethra 16 to function normally. The small diameter connection tube 14 runs down the urethra to the control device 11 which can be conveniently located or mounted on the skin of the user or in the clothes.

FIGS. 11a and b of the drawings show an exemplary embodiment. of the control device in the normal closed position. In this position, the catheter is initially charged (once the catheter is in position) with a fixed volume of saline solution injected via the port 12, using a syringe in the conventional way. This has the effect of fully inflating the locating balloon and closing the hydraulically activated valve located at the distal end of the catheter.

FIGS. 10a and b of the drawings show the control device in the open position. The control device consists of two components: the first is a relatively large diameter sausage shaped tube 18 attached at one end to the connection tube 14 and at the other end to the charging port 12. The tube 18 is made of flexible material e.g. rubber, plastics or the like enabling it to be compressed by the case 19 so that its internal volume is reduced.

The second component is the case 19 comprising a base 20 and lid 19 flexibly hinged 23 and provided with a clip 21 itself flexibly hinged 22 to the lid 19. The tube 18 may be attached to the base 20 of the case by suitable adhesive 29. In use the control device is normally closed and clipped as in FIG. 11a. In this mode the balloon is fully inflated, full hydraulic pressure is in the system and consequently the hydraulic valve at the distal end of the catheter is closed thus preventing the flow of urine.

When urination is required, the clip 21 is released allowing the lid 19 to hinge and the tube 18 to increase its internal volume. This allows saline solution to pass from the locating balloon at the distal end of the catheter via the connection tube 14 lowering the hydraulic pressure in the system. As a result, the hydraulic valve at the distal end opens, as previously explained, allowing normal urination to take place.

Following completion of urination, compressing the lid 19 and re-clipping 21 restores hydraulic pressure in the system thus closing the hydraulic valve at the distal end.

The bulky nature of the opened case serves to remind the user to re-clip the control device as in FIG. 11a following completion of urination.

Referring to FIG. 12 of the drawings, there is shown a longitudinal section through yet another exemplary embodiment of a urinary catheter according to the invention. In this embodiment the urinary catheter comprises a middle tube 2 and an outer tube 1 both of flexible material, forming a closed end. Urine can enter the lumen 3 through an aperture 4 adjacent to the distal end. Over a limited length of catheter, the outer tube wall thickness is reduced 5. The middle and outer tubes are not joined together over the length of reduced wall thickness, but the rest of the middle and outer tubes are substantially bonded together to form a seal such that the reduced thickness area forms an annular space 24. A connecting tube of relatively small diameter 7 cooperates with the annular space 24 between the middle and outer tubes to allow fluid such as saline solution to be pumped into it to form a locating balloon.

A relatively thin additional inner tube 25 is provided which is substantially bonded to the middle tube 2 except over a limited length 26. A second connecting tube 28 of relatively small diameter cooperates with the annular space 27 between the inner and middle tubes to allow fluid, such as saline solution to be pumped into it. The inner and middle tubes and the annular space 27 form a hydraulic valve which, when inflated, closes the lumen 3 thus preventing the flow of urine. In this embodiment the hydraulic valve 26 can be operated independently of the locating balloon 5. The hydraulic valve 26 may be controlled as in the first embodiment by the provision of an external control device connected via the connecting tube 28. In this embodiment the hydraulic valve 26, which forms the annular space 27, is not connected to the locating balloon 5 and its annular space 24. Thus, in this embodiment, the hydraulic valve may be of any size and be located at any convenient position in the lumen.

As with the first embodiment, it will be appreciated that the innermost and outermost tubes 25, 1 need not have a reduced thickness at the inflatable portions 26, 5. Neither do the inflatable portions need to have reduced modulus or be in any other way different from the remainder of the tubes. Having said that, it is desirable that the inflatable portions 26, 5 be of reduced thickness and/or different modulus than the remainder of the tube. Furthermore it may be desirable to form areas, lines-or.points. of weakness in the tubes at the inflatable portions 26, 5 in order to facilitate inflation at an appropriate pressure and, particularly in the case of the innermost tube 26, to ensure that there is complete sealing of the lumen.

The inflation of the two inflatable portions may be achieved, as with the first embodiment, by means of a single source of pressurised fluid. In this case the source would be connected to both connecting tubes 7, 28. Alternatively, these two tubes may be supplied by separate sources of hydraulic or pneumatic pressure.

As shown in FIG. 12, the inflatable portion 26 of the inner tube need not have the same longitudinal extent as the inflatable portion 5 of the outermost tube. It is considered highly desirable for the lumen to be sealed as near to the distal aperture 4 as possible to avoid the creation of a stagnant pool of urine at the distal end of the catheter, which can lead to encrustation and potentially occlusion of the aperture 4 and/or the lumen 3. Accordingly, the internal inflatable portion 26 may extend distally right up to the aperture 4. This of course is also possible with the first embodiment.

In another modification, a further one or more inflatable portions 26 may be provided in the innermost tube 25 to provide a “backup” valve or valves along the length of the catheter. This is also possible with the first embodiment.

In a further modification, the internal valve is provided by an inner tube of shorter length than the whole catheter. For example, the inner tube could comprise an inflatable portion which is not joined to the middle tube 2 in FIG. 12 (or to the outer tube in the first embodiment) and which extends for only a short distance proximally and distally of the inflatable portion, sufficient to allow the innermost tube to be secured in the catheter assembly. In a similar modification, it may be possible to replace this shortened inner tube with a part-tube or “patch” which, for example, extends around only half the circumference of the lumen and which on inflation fills the lumen and seals against the opposite side.

Finally, FIGS. 13 and 14 show an alternative control device. The control device consists of a cylinder body 29 a piston 31 sealed to the cylinder by O′ ring seal 33 and actuated by compression spring 32. A pipe body 30 is attached to the cylinder body 29 by weld 42 or similar and is sealed to the piston body by O′ ring seals 34 and 48. A pin 36 engages with the slot 37 formed in the cylinder body.

FIG. 13 shows the control device in the filling position with the piston 31 fully withdrawn and compressing the spring 32. The pin 36 now in position 47 holds the piston in this position whilst filling takes place. A coloured warning band 41 on the piston barrel indicates that the control device is ready for filling. Filling of the locating balloon is achieved in the normal way using a standard syringe filled with saline, by inserting the syringe tip into the port 35. The port 35 may include a one way valve (not shown). The fluid reaches the locating balloon via pipe 30. A communicating hole 39 allows some of the fluid to fill the cylinder. When the required volume of fluid has been injected into the device, twisting the piston body anticlockwise allows the piston body 31 to move down to the normal valve open position 40 under the control of the pin 36 which would then be in the position 45. Further anticlockwise rotation of the piston body 31 allows the piston body to further move into the cylinder body 29 under the influence of the spring 32 thus displacing the fluid from the cylinder and inflating (closing) the hydraulic balloon valve via tube 38. It will be noted that as soon as the piston body 31 moves into the cylinder body 29 communicating hole 39 becomes closed by seal 34 thus preventing reflux of fluid from the cylinder into the tube 30.

In normal use when the catheter wearer requires to urinate, pulling apart the cylinder body 29 and piston body 31 against the spring 32 draws fluid into the cylinder from the hydraulic valve thus opening the valve and allowing urination to take place. Upon completion of urination releasing the piston body 31 allows the piston 31 to move under the influence of the spring 32 thus forcing fluid in the cylinder to once more flow into the hydraulic valve thus closing it.

If it is required to keep the hydraulic valve permanently open e.g. when it is desired to connect to a urine collection bag overnight then the piston assembly may be withdrawn and rotated either clockwise or anticlockwise to engage the pin 36 into positions 44 or 45 in the slot. The control device may be made of injection moulded plastic components.

The invention has been described above by way of example only. It will be appreciated by persons skilled in the art that many variations and modifications are possible without departing from the scope of the invention as defined by the appended claims.

1. A urinary catheter comprising:
a) a first tube or part-tube;
b) a second tube exterior of and concentric with the first tube or part-tube and joined to it for part of the area of the first tube or part-tube;
c) the first tube or part-tube having a first inflatable portion to which the second tube is not joined;
d) the first inflatable portion being inflatable such as to occlude the lumen of the catheter.

2. A urinary catheter as claimed in claim 1 wherein the second tube has a second inflatable portion which is not joined to the first tube or part-tube and which is inflatable to provide means for retaining the catheter in a bladder.

3. A urinary catheter as claimed in claim 2 wherein the first and second inflatable portions are substantially coincident or overlap.

4. A urinary catheter as claimed in claim 2 wherein the first and second inflatable portions do not overlap.

5. A urinary catheter as claimed in any of claims 2 to 4, further comprising means for supplying pressurised hydraulic or pneumatic fluid to both first and second inflatable portions from the same pressurised source.

6. A urinary catheter as claimed in claim 1 comprising a third tube or part-tube exterior of and concentric with the second tube, to which the second tube is joined for part of the second tube's length, the third tube or part-tube having a third inflatable portion to which the second tube is not joined and which is capable of being inflated to provide means for retaining the catheter in a bladder.

7. A urinary catheter as claimed in claim 2 wherein the first inflatable portion is:
a) substantially coincident with the third inflatable portion;
b) overlaps with the third inflatable portion and, preferably, extends distally of the third inflatable portion; or
c) is located wholly distally of the third inflatable portion.

8. A urinary catheter as claimed in claim 7 further comprising means for supplying pressurised hydraulic or pneumatic fluid for inflation of the first and third inflatable portions either from the same or from separate sources.

9. A urinary catheter as claimed in any of claims 6 to 8 wherein the third inflatable portion is of reduced wall thickness and/or elastic modulus and/or has points, lines or areas of weakness, or is in any other way modified so as to affect the pressure at which it may be inflated compared either to the remainder of the third tube or to the first inflatable portion.

10. A urinary catheter as claimed in any of claims 2 to 9 wherein the first inflatable portion is substantially coincident with, overlaps or is located distally of the third inflatable portion.

11. A urinary catheter as claimed in any preceding claim wherein inflation of the first inflatable portion causes structural collapse of the first inflatable portion and consequent occlusion of the lumen of the catheter.

12. A urinary catheter as claimed in claim 11 wherein reduction in inflation pressure to the first inflatable portion following structural collapse causes the first inflatable portion to re-form at least partially to allow the passage of urine through the lumen of the catheter.

13. A urinary catheter as claimed in claims 11 or 12 wherein the first inflatable portion is provided with one or more scores or points, lines or areas of relative weakness.

14. A urinary catheter as claimed in claim 13 wherein the first inflatable portion is provided with two scores or points, lines or areas of weakness positioned substantially diametrically opposite each other.

15. A urinary catheter as claimed in any preceding claim wherein the first inflatable portion is located so as to allow the lumen of the catheter to be sealed at a point adjacent a distal aperture in the catheter.

16. A urinary catheter as claimed in any preceding claim further comprising an external pressure-adjusting control device and a relative small diameter tube connecting said control device to a region external of the first inflatable portion.

17. A urinary catheter as claimed in claim 15 together with any of claims 2 to 14 wherein the said control device is connected by a relatively small diameter tube to a region exterior of either the second or third inflatable portions.

18. A urinary catheter as claimed in claim 15 or 16 wherein the control device is integrated with a mechanical balloon inflation valve.

19. A urinary catheter as claimed in any of claims 15 to 17 wherein the said control device comprises a compressible case cooperating with a resilient bulb containing pressurised fluid.

20. A urinary catheter as claimed in any preceding claim further comprising a backup seal at or adjacent to the proximal end of the catheter.
